(19) United States
(12) Patent Application Publication
(10) Pub. No.: US 2010/0003319 A1
(43) Pub. Date: Jan. 7, 2010

(54) RALOXIFENE IMMEDIATE RELEASE TABLETS

(75) Inventors: Pavak Mehta, Gujarat (IN); Kamal Mehta, Rajasthan (IN); Abhijeet Upadhye, Maharastra (IN)

Correspondence Address: GLENMARK PHARMACEUTICALS INC USA, 750 CORPORATE DRIVE, MAHWAH, NJ, 07430, US

(73) Assignee: GLENMARK GENERICS LTD., Mumbai-400 (IN)

(21) Appl. No.: 12/494,349

(22) Filed: Jun. 30, 2009

Related U.S. Application Data
(60) Provisional application No. 61/163,487, filed on Mar. 26, 2009.

(30) Foreign Application Priority Data
Jul. 2, 2008 (IN) 1375/MUN/2008

(51) Int. Cl. A61K 9/20; A61K 31/4535; A61K 9/16

(52) U.S. Cl. 424/465; 514/324; 424/489

(57) ABSTRACT

The present invention relates generally to formulations containing raloxifene or pharmaceutical salts thereof, as the active pharmaceutical ingredient and a process for preparing the same.

### PRIORITY

This application claims the benefit to Indian Provisional Application 1375/MUM/2008, filed on Jul. 2, 2008, and under 35 U.S.C. §119 to U.S. Provisional Application 61/163487, filed Mar. 26, 2009, the contents of each of which, are incorporated by reference herein.

### BACKGROUND OF THE INVENTION

1. Technical Field

The present invention relates generally to formulations containing raloxifene or pharmaceutical salts thereof as the active pharmaceutical ingredient and a process for preparing same.

2. Description of the Related Art

Raloxifene, [6-hydroxy-2-(4-hydroxyphenyl)-benzothiophen-3-yl]-[4-[2-(1-piperidyl)ethoxy]phenyl]-methanone, is disclosed in U.S. Pat. No. 4,418,068 and is a selective estrogen receptor modulator and widely used for the treatment and prevention of osteoporosis in postmenopausal women.

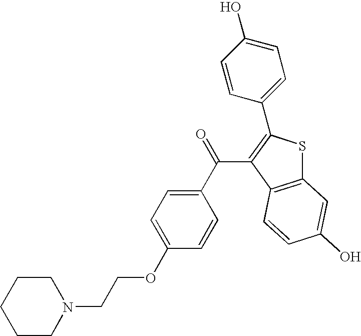

Raloxifene is available under the trade name Evista®. Evista® is also indicated for the reduction in risk of invasive breast cancer in postmenopausal women with osteoporosis and for the reduction in risk of invasive breast cancer in postmenopausal women at high risk of invasive breast cancer.

U.S. Pat. No. 6,458,811 describes pharmaceutical compositions containing raloxifene having a mean particle size less than about 25 μm, wherein about 90% of raloxifene particles have a size of less than about 50 μm.

U.S. Pat. No. 5,811,120 discloses an orally administrable pharmaceutical formulation comprising raloxifene, its esters or ethers, or a pharmaceutically-acceptable salt thereof, in combination with a surfactant, a water-soluble diluent, and optionally a hydrophilic binder.

U.S. Pat. No. 6,894,064 discloses a pharmaceutical composition comprising a) raloxifene in particulate form, said particles having a mean particle size of less than about 25 μm, with at least about 90% of said particles have a size of less than about 50 microns; b) a surfactant; and c) a water-soluble diluent.

U.S. Patent Publication 2005/0008704 (the '704 patent) describes a pharmaceutical composition of raloxifene having an enhanced solubility. The '704 patent publication discloses a composition comprising raloxifene and polyethylene glycol, wherein the ratio of polyethylene glycol to raloxifene by weight is from about 0.2:1 to about 10:1, and the polyethylene glycol has a melting point of at least 37° C.

### SUMMARY OF THE INVENTION

The present invention provides raloxifene or a pharmaceutical salt thereof, having a mean particle size of at least about 25 μm.

The present invention provides a pharmaceutical composition comprising a therapeutically effective amount of raloxifene or a pharmaceutically acceptable salt thereof having a mean particle size of at least about 25 μm.

The present invention provides a pharmaceutical composition comprising a therapeutically effective amount of raloxifene or a pharmaceutically acceptable salt thereof having a mean particle size of at least about 25 μm and a pH modifier.

The present invention provides a pharmaceutical composition comprising a therapeutically effective amount of raloxifene or a pharmaceutically acceptable salt thereof having a mean particle size of at least about 25 μm and a solubilizer.

The present invention provides a process for a preparation of dosage forms of raloxifene or a pharmaceutically acceptable salt thereof having a mean particle size of at least about 25 μm for oral administration.

In another aspect, the present invention provides a process for the preparation of a pharmaceutical composition comprising raloxifene or a pharmaceutically acceptable salt thereof, having a mean particle size of at least about 25 μm, that involves (a) blending the raloxifene or a pharmaceutically acceptable salt thereof, having a mean particle size of at least about 25 μm and one or more pharmaceutical acceptable excipients, and compacting the blend; (b) milling the compacted blend for one or more times; (c) granulating the milled compacts with a solution or a dispersion comprising at least one from the group of binder, surfactant, pH modifier (c) drying and mixing the sized granules with one or more pharmaceutically acceptable excipients and compressing the mixture into tablets.

### DETAILED DESCRIPTION OF THE PREFERRED EMBODIMENTS

The present invention relates generally to compositions containing raloxifene or pharmaceutical salts thereof as the active pharmaceutical ingredient and a process for preparing same.

The composition of the present invention uses raloxifene or pharmaceutically acceptable salts thereof, having a mean particle size of at least about 25 μm.

The pharmaceutical composition of the present invention comprises raloxifene or pharmaceutically acceptable salts thereof, of mean particle size between, 25 μm to 125 μm, preferably between 30 μm to 100 μm, and more preferably between 30 μm to 50 μm.

The pharmaceutical composition of the present invention comprises raloxifene or pharmaceutically acceptable salts thereof, diluents, binders, disintegrants, lubricants and optionally pH modifiers, solubilizing agents, glidants, coloring agents, flavoring agents, sweeteners.

The pharmaceutical composition of present invention comprises raloxifene or pharmaceutically acceptable salts thereof, 5 to 35% diluents from about 20% to 70%, and disintegrants from about 1% to 20% and optionally lubricants and optionally pH modifiers, solubilizing agents, glidants, coloring agents, flavoring agents, sweeteners.

Suitable pH modifiers include, but are not limited to, basifying agents, acidifying agents and the like and mixtures thereof. Examples of pH modifiers for use herein include meglumine, magnesium oxide, calcium carbonate, fumaric acid, succinic acid, citric acid and the like, preferably the organic base, meglumine, which is both a pH adjusting agent and solubilizing agent.

Suitable carriers or diluents are selected from starch, saccharides, microcrystalline cellulose, other cellulose derivatives, calcium phosphate, sodium calcium phosphate (NaCaPO₄), sugar alcohol, lactose and mixtures thereof.

Suitable binders, which are used to impart cohesive properties to the pellets or granules, include water soluble and insoluble binders. It is preferred to use water soluble binder selected from cellulose derivatives, sugars, gums, gelatin, povidone, pregelatinized starch, sugar solution, and polyvinyl alcohol. The most preferred water soluble binder is hydroxypropyl methylcellulose (HPMC) of different viscosity grades, where the preferred viscosity grades of HPMC are 3 cps to 10cps.

Suitable disintegrating agents, which are the substances or mixtures of substances added to a pellet to facilitate its breakup or disintegration after administration, are selected from modified or unmodified starches, clays, cross-linked polyvinylidenepyrrolidone (PVP), modified or unmodified celluloses.

It is also preferred, but not necessary, that the dosage form can comprise a small amount of a lubricant such as, for example, magnesium stearate.

The choice of excipients is not limited to the example disclosed herewith; it may include any suitable excipients mentioned in the Handbook of Pharmaceutical Excipients (5^{th} ed.).

The dosage form of raloxifene or its pharmaceutical salt can be prepared by tablet press, roller compactor or any other machine or machine aid used to prepare solid dosage forms, known in the art.

One embodiment of the present invention provides formulations in the form of film coated tablets, comprising the active pharmaceutical ingredient raloxifene or pharmaceutical salts thereof and other required pharmaceutically acceptable excipients and a process for its preparation.

Suitable film coatings include cellulose derivatives such as hydroxypropyl cellulose, hydroxypropyl methyl cellulose, hydroxyethyl cellulose, methyl hydroxyethyl cellulose. HPMC is preferred film coating agent.

The process for preparing the composition of raloxifene or its pharmaceutically acceptable salt involves, uniformly mixing raloxifene or its pharmaceutically acceptable salt with one or more fillers, and disintegrants, followed by the roll compaction of the dry blend, milling the compacts for one or more times through suitable screen, preferably 0.25 mm screen, granulating the compacts in high shear mixers, using suitable solvents. The granules are dried and reduced to the desired size, blended with the disintegrant and then lubricated. The lubricated blend is further compressed to tablets. If desired, the tablets can be coated with rapidly disintegrating coating composition.

The examples that follow are provided to enable one skilled in the art to practice the invention and are merely illustrative of the invention. The examples should not be read as limiting the scope of the invention as defined in the claims. The percent w/w formula compositions of raloxifene or its pharmaceutical salt, in particular, raloxifene hydrochloride (HCl) are set forth in Examples I and II.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

### EXAMPLES

### Part A: Preparation of Raloxifene Tablets

### Example I

| | Ingredient | | |
|---|---|---|---|
| Sr. | Roller Compaction and | Qty/Tab. (mg) | Function of |
| No. | Milling | Strengths (60 mg) | Excipients |
| 1. | Raloxifene hydrochloride | 60.00 | API |
| 2. | Lactose Monohydrate | 120.00 | Diluent |
| 3. | Crospovidone | 5.00 | Disintegrant |
| 4. | Microcrystalline Cellulose | 20.00 | Diluent |

Base intragranular ingredients

| | | | |
|---|---|---|---|
| 5. | Crospovidone | 2.50 | Disintegrant |
| 6. | HPMC-3 cps | 5.00 | Binder |
| 7. | Poloxamer-407 | 1.25 | Surfactant |

| | | | |
|---|---|---|---|
| 8. | Purified Water | q.s. | Granulating fluid |

Extragranular ingredients

| 9 | Lactose Monohydrate | 28.75 | | Diluent |
|---|---|---|---|---|
| 10 | Crospovidone 10 | 5.00 | | Disintegrant |
| 11 | Magnesium Stearate | 2.50 | | Lubricant |
| Total weight of core tablet | | 250.00 | | |
| 12 | Opadry-OY-LS-28908 | 7.5 | mg | Coating Agent |

| | | |
|---|---|---|
| 13 | Purified water | q.s. |

| | |
|---|---|
| Total weight of coated Tablet | 257.5 |

Process: Raloxifene hydrochloride (HCl), microcrystalline cellulose, lactose and crospovidone were shifted and uniformly blended; the blend was compacted by using roller compactor and the compacts were milled in a comminuting mill. Pass the milled blend through mesh #30 & mix with crospovidone; granulate this mixture with the clear binder solution of poloxamer 407 and hydroxypropylmethylcellulose (HPMC)-5 cPs in purified water. Dry the granules in a fluid bed dryer until the % limit of detection (LOD) of not more than (NMT) 2.0% w/w is attained and the granules are screened through mesh #25. Uniformly mix the dried screened granules with crospovidone and lactose monohydrate and lubricate this blend by using magnesium stearate. The lubricated blend was then compressed into tablets using suitable tooling.

### Example II

| | Ingredient | | |
|---|---|---|---|
| Sr. | Roller Compaction and | Qty/Tab. (mg) | Function of |
| No. | Milling | Strengths (60 mg) | Excipients |
| 1. | Raloxifene HCl | 60.00 | API |
| 2. | Lactose Monohydrate | 120.00 | Diluent |
| 3. | Crospovidone | 5.00 | Disintegrant |
| 4. | Microcrystalline Cellulose | 20.00 | Diluent |

Base intragranular ingredients

| | | | |
|---|---|---|---|
| 5. | Crospovidone | 2.50 | Disintegrant |
| 6. | HPMC-3 cps | 5.00 | Binder |
| 7. | Poloxamer-407 | 1.25 | Surfactant |
| 8. | Citric Acid | 5.00 | Acidifying Agent |

| | | | |
|---|---|---|---|
| 9. | Purified Water | q.s. | Granulating fluid |

Extragranular ingredients

| 10. | Lactose Monohydrate | 23.75 | | Diluent |
|---|---|---|---|---|
| 11. | Crospovidone | 5.00 | | Disintegrant |
| 12. | Magnesium Stearate | 2.50 | | Lubricant |
| Total weight of core tablet | | 250.00 | | |
| 13. | Opadry-OY-LS-28908 | 7.5 | mg | Coating Agent |

| | | |
|---|---|---|
| 14. | Purified water | q.s. |

| | |
|---|---|
| Total weight of coated Tablet | 257.5 |

Process: The process follows largely Example I. However, citric acid is added at the wet granulation step, dissolved in granulation solution along with poloxamer 407 and HPMC 3cPs.

### Part B: Comparative Dissolution Studies

The dissolution profile of the tablets, prepared as in Examples I-II, was undertaken in a 1000 ml of 0.1% polysorbate-80 in water at 37° C. using USP Dissolution Apparatus Type II at an agitation of 50 rpm. The dissolution profile of the Evista® tablets was carried out under the same conditions for comparison.

| Time | Dissolution profile of | Dissolution profile of In-house |
|---|---|---|
| (min) | Evista ® | formulation |
| 5 | 18 | 68 |
| 10 | 53 | 84 |
| 20 | 86 | 88 |
| 30 | 92 | 91 |
| 45 | 95 | 93 |

The data obtained, as shown above, from the dissolution studies shows that the formulations of raloxifene, prepared as described herein, release more than 85% raloxifene within 30 minutes, which is comparable to the dissolution rate of the marketed formulation (Evista®). The dissolved raloxifene is expressed in cumulative percent of drug dissolved over an elapsed time period in minutes. The formulations of the present invention use raloxifene HCl having mean particle size at least about 25 μm.

As raloxifene is very slightly soluble in water, attempts have been made to enhance the dissolution rate and therefore the availability and bioavailability of raloxifene, by reducing the particle size of the pure raloxifene, or by using solubilizing agents (surfactants).

The '811 patent, for example, discloses that the desired dissolution and bioavailability characteristics or raloxifene is gained with the control of particle size to a narrow range.

While the '120 patent discloses that the use of water soluble binder, water soluble diluent and surfactant overcome the limitations of bioavailability of raloxifene.

The dissolution data in PART B above (after EXAMPLES) shows that raloxifene tablets, as herein described and prepared above, using unmicronized raloxifene API, give the desired dissolution profile as compared to the marketed tablet (Evista®).

The formulation of raloxifene or a pharmaceutical salt thereof and a process of making the same, as herein described above, advantageously avoids the micronization of raloxifene API, which requires special equipments and strict controls.

We claim:

1. A pharmaceutical composition comprising raloxifene or a pharmaceutically acceptable salt thereof, of a mean particle size of at least about 25 μm.

2. The composition of claim 1, comprising raloxifene or pharmaceutically acceptable salts thereof, of mean particle size between, 25 μm to 125 μm,

3. The composition of claim 1, comprising raloxifene or pharmaceutically acceptable salts thereof, of mean particle size between 30 μm to 100 μm.

4. The composition of claim 1, comprising raloxifene or pharmaceutically acceptable salts thereof, of mean particle size between, 30 μm to 50 μm.

5. A pharmaceutical composition, comprising raloxifene or pharmaceutically acceptable salts thereof of mean particle size of at least about 25 μm, at least one filler, a disintegrant and a lubricant and optionally a solubilizing agent and pH modifier

6. The composition of claim 5, comprising raloxifene or pharmaceutically acceptable salts thereof, of mean particle size between 30 μm to 100 μm, at least one filler, a disintegrant and a lubricant and optionally a solubilising agent and pH modifier.

7. The composition of claim 6, comprising raloxifene or pharmaceutically acceptable salts thereof, of mean particle size between 30 μm to 50 μm, at least one filler selected from lactose, microcrystalline cellulose, a disintegrant selected from crospovidone, croscarmellose sodium, a lubricant and optionally a solubilising agent and pH modifier.

8. The composition of claim 7, wherein the solubilising agent is selected from poloxamers and sodium lauryl sulphate.

9. The composition of claim 7, wherein the pH modifier is an organic acid selected from the group comprising of citric acid, malic acid and tartaric acid.

10. A pharmaceutical composition comprising raloxifene or pharmaceutically acceptable salts thereof, of mean particle size of at least about 25 μm, prepared by roller compaction.

11. The composition of claim 10, prepared by a process comprising (a) roller compaction of raloxifene or a pharmaceutically acceptable salt thereof with one or more pharmaceutically acceptable excipient(s); (b) milling the compacted mass for one or more times by a comminuting mill to obtain the granules.

12. The composition of claim 10, prepared by a process comprising (a) roller compaction of raloxifene or a pharmaceutically acceptable salt thereof with one or more pharmaceutically acceptable excipient (s); (b) milling the compacted mass for one or more times by comminuting mill for two to four times; (c) granulating the milled granules with a solution or a dispersion comprising at least one from the group of binder, surfactant, pH modifier ;(d) drying the wet granules (e) mixing the dried granules with lubricant; (f) compressing the mixture into tablets.
